(19) United States
(12) Patent Application Publication
(10) Pub. No.: US 2025/0032375 A1
(43) Pub. Date: Jan. 30, 2025

(54) SOLVENT MIXTURES COMPRISING 1,3-BUTYLENE GLYCOL AND TRIETHYL-CITRATE

(71) Applicant: Givaudan SA, VERNIER (CH)

(72) Inventors: Agnes BOMBRUN, Kemptthal (CH); Chiara PICCOLO, Kemptthal (CH); Sandrine PIANACCI, Paris (FR)

(21) Appl. No.: 18/833,618

(22) PCT Filed: Jan. 30, 2023
(86) PCT No.: PCT/EP2023/052122

§ 371 Date: Jul. 26, 2024

(30) Foreign Application Priority Data
Jan. 31, 2022 (GB) 2201247.0
Aug. 2, 2022 (GB) 2211235.3

(51) Int. Cl. A61K 8/34; A61K 8/37; A61Q 13/00

(52) U.S. Cl. CPC A61K 8/345; A61K 8/37; A61Q 13/00; A61K 2800/30

(57) ABSTRACT

The invention relates to a solvent mixture consisting of 1,3-butylene glycol and triethyl citrate that is a suitable solvent in perfumery and can be derived from natural and renewable resources.

### TECHNICAL FIELD

The present invention relates generally to solvents or diluents useful in perfumery, in particular to solvents or diluents obtainable from natural and/or renewable resources.

### BACKGROUND

Solvents or diluents in perfumery need to fulfil several criteria. They should be able to solubilize or dilute preferably all perfumery compounds and have substantially no negative contribution to the odour. Furthermore, they should be safe for human use and for the environment. In addition, they should allow the fragrance compound(s) dissolved therein to show its physical and performance characteristics. Finally, they should allow the incorporation of the fragrance compound(s) or the fragrance composition dissolved therein into a consumer product.

For example, dipropylene glycol (DPG, CAS No. 25265-71-8) is a known and widely used solvent in perfumery. It is colorless and almost odorless, which allows for broad applications in all categories. DPG is a synthetic solvent.

Alternative solvents, for example benzyl benzoate or isopropyl myristate, could be derived from natural sources; however, they might have the disadvantage to not solubilize all ingredients used in perfumery. Furthermore, they might have a significant color and/or odour and cause stability issues.

So there remains a need for naturally derived solvents or solvent mixtures with good solubilizing properties. In particular, there remains a need for naturally derived solvents or solvent mixtures as replacer for DPG.

### SUMMARY

In accordance with a first aspect of the present invention there is provided a solvent mixture comprising 1,3-butylene glycol and triethyl citrate.

In accordance with a second aspect of the present invention there is provided a fragrance composition comprising one or more fragrance compounds and the solvent mixture.

In accordance with a third aspect of the present invention there is provided the use of the solvent mixture as solvent for a fragrance compound and/or fragrance composition.

In accordance with a fourth aspect of the present invention there is provided the use of the fragrance composition comprising one or more fragrance compounds and the solvent mixture for perfuming consumer products.

In accordance with a fifth aspect of the present invention there is provided a consumer product comprising the fragrance composition comprising one or more fragrance compounds and the solvent mixture, and a consumer product base.

In accordance with a sixth aspect of the present invention there is provided a method to confer, enhance, improve or modify the hedonic properties of a consumer product, the method comprising the addition of the fragrance composition comprising one or more fragrance compounds and the solvent mixture to said consumer product.

Certain embodiments of any aspect of the present invention may provide one or more of the following advantages:
the solvent mixture can be derived from renewable resources;
it is biodegradable;
it has good solubility properties for a broad palette of individual fragrance compounds and/or fragrance compositions;
it is nearly odourless;
it is colourless;
it is stable over time and under different conditions (for example temperature, light).

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### DETAILED DESCRIPTION

The present invention is based on the surprising finding that a mixture of two specific solvents shows excellent solubility properties towards fragrance ingredients. In particular, the mixture of said two solvents outperforms the individual solvents.

There is therefore provided herein a solvent mixture consisting of 1,3-butylene glycol and triethyl citrate.

Said solvent mixture is a suitable solvent for fragrance compounds and/or fragrance compositions. It has good solubility properties for individual fragrance compounds and/or fragrance compositions. It is nearly odourless and colourless. Fragrance compounds and/or fragrance compositions dissolved therein are stable over time and under different conditions like elevated temperature and/or light exposure.

For example, said solvent mixture can replace DPG.

It can be obtained from natural sources and is biodegradable.

1,3-butylene glycol (CAS No. 107-88-0) can be derived from natural sources. For example, it can be obtained from renewable plant sugars in a fermentation process, as described in WO2010127319A2. 1,3-butylene glycol is colorless and nearly odourless, having a faint sweet note. 1,3-butylene glycol is biodegradable. It is a chiral compound that exists as R-isomer (CAS No. 6290-03-5) and S-isomer (CAS No. 24621-61-2). It can be used as racemic mixture, as pure enantiomer, or enriched in one of the two isomers.

Triethyl citrate (TEC, CAS No. 77-93-0) is colorless and odourless. It can be derived from natural sources, for example via fermentation of ethanol and natural citric acid. It is readily biodegradable and considered to have low toxicity.

The term “derived from natural/renewable resources” refers to a material that can be derived from a raw material, which is produced or manufactured in a finite period to replace the portion of said raw material that has been consumed.

The term “biodegradable” refers to a compound that can be degraded by microorganisms, i.e. broken down into smaller components that substantially do not represent a negative impact on the environment.

1,3-butylene glycol and triethyl citrate are low environmental impactful and therefore sustainable materials, each having a low carbon footprint of the renewable raw material and of the manufacturing process, which is entailing low energy demanding biotechnological steps, and being biodegradable.

In one specific embodiment the ratio of 1,3-butylene glycol and triethyl citrate in the solvent mixture is between 1:5 and 5:1.

For example, the ratio of 1,3-butylene glycol and triethyl citrate in the solvent mixture is between 1:4 and 4:1, or between 1:3 and 3:1, and in a specific embodiment the ratio is about 1:1.

In one embodiment, there is provided a solvent mixture consisting of 1,3-butylene glycol and triethyl citrate in a ratio between 1:4 and 4:1. For example, the solvent mixture consists of 1,3-butylene glycol and triethyl citrate in a ratio of about 1:4.

In one specific embodiment there is provided a solvent mixture comprising 1,3-butylene glycol and triethyl citrate, which means that the solvent mixture consisting of 1,3-butylene glycol and triethyl citrate is admixed with a further compound. The ratio of 1,3-butylene glycol and triethyl citrate is between 1:4 and 4:1.

The solvent mixture comprising or consisting of 1,3-butylene glycol and triethyl citrate is also suitable for applications further comprising water.

For example, the amount of water in the solvent mixture with a fragrance compound and/or fragrance composition dissolved therein may be up to 5% by weight of the resulting composition, or up to 10% by weight of the resulting composition.

The solvent mixture comprising or consisting of 1,3-butylene glycol and triethyl citrate is a suitable solvent for a fragrance compound and/or fragrance composition.

The term “individual fragrance compound” or “fragrance compound” means a single or specific odourant molecule, which is a compound having a smell. Such fragrance compounds are described, for example, in “Perfume and Flavor Chemicals”, S. Arctander, Ed., Vol. I & II, Allured Publishing Corporation, Carol Stream, USA, 2003 and include fragrance compounds of natural or synthetic origin and fragrance ingredients like essential oils.

The term “fragrance composition” or “perfume composition” means at least a mixture of fragrance compounds, also referred to as perfume mixture or an accord. The fragrance composition may further comprise one or more ingredients or excipients conventionally used in conjunction with odourants in perfume compositions, for example a carrier material and/or an auxiliary agent.

As used herein, “carrier material” means a material, which is practically neutral from a odourant point of view, i.e. a material that does not significantly alter the organoleptic properties of odourants. A solvent or a diluent is an example of such carrier material.

The term “auxiliary agent” refers to ingredients that might be employed in a fragrance composition for reasons not specifically related to the olfactive performance of said composition. For example, an auxiliary agent may be an ingredient that acts as an aid to processing a fragrance ingredient or ingredients, or a composition containing said ingredient(s), or it may improve handling or storage of a fragrance ingredient or composition containing same. It might also be an ingredient that provides additional benefits such as imparting color or texture. It might also be an ingredient that imparts light resistance or chemical stability to one or more ingredients contained in a perfume composition. A detailed description of the nature and type of adjuvants commonly used in perfume compositions containing same cannot be exhaustive, but it has to be mentioned that said ingredients are well known to a person skilled in the art.

For example, the perfume composition may comprise an anti-oxidant adjuvant. Said anti-oxidant may be selected from carnosic acid (for example in the form of an extract from a plant of the Lamiaceae family, like rosemary), Tinogard® TT (BASF), Tinogard® Q (BASF), Tocopherol (including its isomers, CAS 59-02-9; 364-49-8; 18920-62-2; 121854-78-2), 2,6-bis(1,1-dimethylethyl)-4-methylphenol (BHT, CAS 128-37-0) and related phenols, hydroquinones (CAS 121-31-9).

In general, solvent mixtures of 1,3-butylene glycol with other solvents are of interest. The other solvents to be mixed with 1,3-butylene glycol can be natural, nature identical, naturally derived or synthetic. A natural product is a chemical compound or substance produced by a living organism that is found in nature. A nature identical substance has been prepared synthetically but is chemically identical to a natural one. Naturally derived compounds are any ingredient whose starting material originates from plants, minerals, microbes or animals that may be adapted to provide performance.

The is a particular interest to mix 1,3-butylene glycol with other solvents that are natural or naturally derived from renewable resources, and/or are biodegradable.

For example, the other solvent that is natural or naturally derived from renewable resources can be selected from the group consisting of hydrogenated methyl rosinate (for example Hercolyn, Hercolyn DW, CAS No. 8050-15-5), Ethanol (for example Ethyl alcohol (CN), ethanol fine undenaturated 192 proof, perfumery grade, CAS No. 64-17-5), Glycerine (for example Glycerine CP USP 99.5% white, CAS No. 56-81-5), Wickenol 163 (mixture of Diethylhexyl Adipate, Ethylhexyl Palmitate and Ethylhexyl Stearate), Lipovol Safflower Oil (Carthamus tinctorius (Safflower) Seed Oil), Isopropylidene glycerol (for example Solketal, CAS No. 100-79-8), diethyl citrate, methyl soyate (CAS No. 67784-80-9), acetyl tributyl citrate (CAS No. 77-90-7), isopropyl myristate (CAS No. 110-27-0), water, medium chain triglyceride, castor seed oil, and others.

The resulting solvent mixture can be obtained from renewable resources and/or is biodegradable.

For specific applications, a combination of 1,3-butylene glycol with solvents that are obtained from other resources might be useful. Some of the other solvents might be biodegradable. Said other solvents can be selected from the group consisting of isoparaffinic hydrocarbon (for example Isopar L, Isopar H, Isopar M), Dipropylene glycol dimethyl ether (for example Dowanol DPM glycol ether, CAS No. 111109-77-4), isopropyl alcohol (CAS No. 67-63-0), methyl-3 methoxy-3 butanol (CAS No. 56539-66-3), Dioctyl adipate (CAS No. 123-79-5), Tripropylene glycol methyl ether (for example Dowanol TPM glycol ether, CAS No. 25498-49-1), Dipropylene Glycol n-Propyl Ether (for example Dowanol DPnP glycol ether, CAS No. 29911-27-1), Dibasic Ester (low vapor pressure), ethylene diglycol monoethyl ether (eg. Carbitol Cosmetic, CAS No. 111-90-0), benzyl benzoate (CAS No. 120-51-4), Glyceryl diacetate (Diacetin, CAS No. 25395-31-7), 2-Methylpentane-2,4-diol (Diolane, CAS No. 107-41-5), Dipropylene Glycol (CAS No. 25265-71-8), Di(propylene glycol) methyl ether acetate (eg. Dowanol DPMA, CAS No. 88917-22-0), Propylene Glycol n-Butyl Ether (eg. Dowanol PnB, CAS No. 5131-66-8), propylene glycol (CAS No. 57-55-6), Triacetin (CAS No. 102-76-1), diethyl phthalate (CAS No. 84-66-2), polyethylene glycol 400, propylene glycol methyl ether (eg. Dowanol PM, CAS No. 107-98-2), and others.

The resulting solvent mixture can be partially obtained from renewable resources and/or is biodegradable.

In another specific embodiment, there is provided a fragrance composition comprising one or more fragrance compounds and the solvent mixture according to the invention, that is a solvent mixture comprising or consisting of 1,3-butylene glycol and triethyl citrate. Said fragrance composition is provided in a solvent that is derived from natural and renewable resources.

In a further embodiment, there is provided the fragrance composition comprising one or more fragrance compounds and the solvent mixture according to the invention, that is essentially free of dipropylene glycol (DPG). Said fragrance composition consists mainly of one or more fragrance compounds, 1,3-butylene glycol and triethyl citrate. “Essentially free of DPG” means, that the amount of dipropylene glycol in said fragrance composition is limited, for example to an amount of 1% by weight of the fragrance composition or less. For example, a minor amount of DPG might be present in the fragrance composition if one or more of the fragrance compounds are provided in diluted form which is comprising DPG.

In a further embodiment, there is provided the fragrance composition consisting essentially of one or more fragrance compounds and the solvent mixture according to the invention and ethanol. Said fragrance composition consists mainly of one or more fragrance compounds, 1,3-butylene glycol, triethyl citrate and ethanol. The amount of dipropylene glycol in said fragrance composition is limited, for example to an amount of 1% by weight of the fragrance composition or less.

In a further embodiment, there is provided the use of the solvent mixture comprising or consisting of 1,3-butylene glycol and triethyl citrate as solvent for a fragrance compound and/or fragrance composition.

There is further provided the use of the fragrance composition comprising one or more fragrance compounds and the solvent mixture comprising or consisting of 1,3-butylene glycol and triethyl citrate for perfuming consumer products.

The solvent mixture of the present invention allows the incorporation of the fragrance compound(s) or the fragrance composition dissolved therein into a consumer product.

In a further embodiment, there is provided the consumer product comprising the fragrance composition comprising one or more fragrance compounds and the solvent mixture according to the invention and a consumer product base.

The consumer product for example is selected from fine fragrance (perfume, eau de parfume, eau de toilette, eau de Cologne and others), personal care products (body care products, hair care products, cosmetic products), fabric care products, home care products and air care products. As used herein, “consumer product base” means a composition for use as a consumer product to fulfill specific actions, such as cleaning, softening, and caring or the like.

The consumer product base may contain further solvents, which depend on the nature of the consumer product. The base of the consumer product might also contain water, for example in amounts about 15% by weight of the consumer product. The solvent mixture of the present invention is used to overcome solubility issues between the water and the fragrance composition.

Fine fragrance products include perfume, eau de parfume, eau de toilette, eau de Cologne and others. Those products differ in the concentration of the fragrance composition, ethanol and water.

Personal care products include for example all kinds of body care products. Especially interesting products are hair care products, for example shampoos, conditioners and hairsprays, and skin care products, like lotions or creams, or soaps, bath and shower gels and deodorants, or cosmetic products.

Home care products include all kinds of detergents, window cleaners, hard surface cleaners, all-purpose cleaners and furniture polishes. Preferably, the products are liquids, e.g. fabric detergent or conditioner compositions.

The solvent of the present invention is suitable for incorporation of the fragrance composition into consumer product bases with or without water.

In a further embodiment, there is provided the method to confer, enhance, improve or modify the hedonic properties of a consumer product, the method comprising the addition of a fragrance composition comprising one or more fragrance compounds and the solvent mixture according to the invention to said consumer product.

The resulting consumer product contains a perfume composition which solvent is derived from natural and renewable sources and is biodegradable.

### EXAMPLES

### General

### Abbreviations

MIX: mixture of 1,3-butylene glycol and triethyl citrate in a given ratio by weight.

### Example 1: Single Ingredients Solubility

The solubility of fragrance compounds and/or fragrance compositions has been assessed by a) visual inspection (soluble—clear solution; non soluble—turbid solution or phase separation); and b) absorption spectrometry (Spectrophotometer, WPA biowave, 008000 Cell Density Meter, light absorbance of 1 cm path length of sample at 600 nm in AU absorption units (arbitrary unit) is measured. If the sample is dissolved, there is low absorbance (close to zero), any turbidity (>0.5) indicates incomplete solution.

### a) Peonile (2-cyclohexylidene-2-phenylacetonitrile, CAS-No. 10461-98-0)

| | 10% | 10% in 1,3- | 10% | 10% | 10% |
|---|---|---|---|---|---|
| | in | butylene | in 1:1 | in 1:3 | in 3:1 |
| | TEC | glycol | MIX | MIX | MIX |
| visual | soluble | non soluble | soluble | soluble | soluble |
| inspection | | | | | |
| ultrasonic bath, | 0.02 | >2 | 0.00 | 0.02 | 0.02 |
| spectrometer | | | | | |

### b) Heliotropine Crist (1-naphthalen-2-ylethanone, CAS-No. 93-08-3)

| | | 10% in | | | | |
|---|---|---|---|---|---|---|
| | | 1,3- | | 10% in | 10% in | 50% in |
| | 10% in | butylene | 10% in | 1:3 MIX | 3:1 MIX | 1:3 MIX |
| | TEC (*) | glycol (*) | 1:1 MIX | (*) | (*) | (*) |
| visual inspection | soluble | soluble | soluble | soluble | soluble | soluble |
| ultrasonic bath, | 0.01 | 0.03 | 0.00 | 0.00 | 0.02 | 0.02 |
| spectrometer | | | | | | |

(*) The sample was heated and cooled down to room temperature.

### c) Coumarine Pure Crist (1-benzopyran-2-one, CAS No. 91-64-5)

| | 10% | 10% in 1,3- | 10% | 10% | 10% |
|---|---|---|---|---|---|
| | in | butylene | in 1:1 | in 1:3 | in 3:1 |
| | TEC (*) | glycol (*) | MIX (*) | MIX (*) | MIX (*) |
| visual | soluble | soluble | soluble | soluble | soluble |
| inspection | | | | | |
| ultrasonic bath, | 0.07 | 0.06 | 0.05 | 0.00 | 0.02 |
| spectrometer | | | | | |

(*) The sample was heated and cooled down to room temperature.

### d) Benzyl Benzoate (CAS No. 120-51-4)

| | 10% in | 10% in 1,3- | 10% in | 10% in |
|---|---|---|---|---|
| | TEC | butylene glycol | 1:1 MIX | 1:3 MIX |
| visual | soluble | non soluble | soluble | soluble |
| inspection | | | | |
| ultrasonic bath, | 0.03 | >2 | 0.03 | 0.03 |
| spectrometer | | | | |

### e) Ethyl Vanilline (3-Ethoxy-4-hydroxybenzaldehyde, CAS No. 121-32-4)

| | | 10% in | | | 30% in | |
|---|---|---|---|---|---|---|
| | | 1,3- | 10% in | | 1,3- | 30% in |
| | 10% in | butylene | 1:1 MIX | 30% in | butylene | 1:1 MIX |
| | TEC (*) | glycol (*) | (*) | TEC (*) | glycol (*) | (*) |
| visual inspection | soluble | soluble | soluble | non | non | soluble |
| | | | | soluble | soluble | |
| ultrasonic bath, | 0.06 | 0.01 | 0.00 | solid | solid | 0.00 |
| spectrometer | | | | | | |

| | 40% in | 10% in | 20% in | 10% in | 20% in |
|---|---|---|---|---|---|
| | 1:1 MIX | 1:3 MIX | 1:3 MIX | 3:1 MIX | 3:1 MIX |
| | (*) | (*) | (*) | (*) | (*) |
| visual inspection | soluble | soluble | soluble | soluble | soluble |
| ultrasonic bath, | 0.04 | 0.00 | 0.00 | 0.00 | 0.01 |
| spectrometer | | | | | |

(*) The sample was heated and cooled down to room temperature.

### f) Ylang Ylang Ess Extra Comores Orpur (CAS No. 8006-81-3)

| | 1% in | 1% in 1,3- | 1% in | 1% in |
|---|---|---|---|---|
| | TEC | butylene glycol | 1:1 MIX | 1:3 MIX |
| visual | soluble | non soluble | soluble | soluble |
| inspection | | | | |
| ultrasonic bath, | 0.02 | 0.54 | 0.07 | 0.11 |
| spectrometer | | | | |

### g) Maltol Lg Crist (3-hydroxy-2-methyl pyran-4-one, CAS No. 118-71-8)

| | | 1% in 1,3- | | |
|---|---|---|---|---|
| | 1% in | butylene | 1% in 1:3 | 1% in 3:1 |
| | TEC (*) | glycol (*) | MIX (*) | MIX (*) |
| visual | non soluble | non soluble | soluble | soluble |
| inspection | | | | |
| ultrasonic bath, | solid | solid | 0.02 | 0.02 |
| spectrometer | | | | |

(*) The sample was heated and cooled down to room temperature.

### Example 2: Fragrance Composition Solubility

### a) Raspberry Accord

| | | | parts by |
|---|---|---|---|
| CAS-No. | ingredient name | chemical name | weight |
| 88-41-5 | Agrumex | 2-tert-butylcyclohexyl acetate | 20 |
| 28645-51-4 | Ambrettolide | Oxacycloheptadec-10-en-2-one | 40 |
| 6790-58-5 | Ambrofix | (3aR,5aS,9aS,9bR)-3a,6,6,9a- | 20 |
| | | tetramethyl-2,4,5,5a,7,8,9,9b- | |
| | | octahydro-1H-benzo[e][1]benzofuran | |
| 140-11-4 | Benzyl acetate | Benzyl acetate | 20 |
| 150-84-5 | Citronellyl acetate | 3,7-dimethyl-6-octen-1-yl acetate | 60 |
| 10339-55-6 | Ethyl linalool | (6E)-3,7-dimethylnona-1,6-dien-3-ol | 60 |
| 4940-11-8 | Ethyl maltol | 3-hydroxy-2-ethyl-4H-pyran-4-one | 10 |
| 121-32-4 | Ethyl vanillin | 4-hydroxy-3-ethoxybenzaldehyde | 60 |
| 24851-98-7 | Hedione | Methyl 3-oxo-2- | 80 |
| | | pentylcyclopentaneacetate | |
| 142-92-7 | Hexyl acetate | Hexyl acetate | 40 |
| 79-77-6 | lonone beta | 4-(2,6,6-trimethyl-1-cyclohexen-1-yl)- | 30 |
| | | 3-buten-2-one | |
| 54464-57-2 | Iso e super | 1-(2,3,8,8-tetramethyl-1,3,4,5,6,7- | 40 |
| | | hexahydronaphthalen-2-yl)ethanone | |
| 1335-46-2 | Isoraldeine 70 | 1-(2,6,6-trimethyl-1-cyclohex-2- | 60 |
| | | enyl)pent-1-en-3-one N- | |
| 115-95-7 | Linalyl acetate synthetic | 3,7-dimethyl-1,6-octadien-3-yl acetate | 40 |
| 329925-33-9 | Nirvanolide | (10Z)-13-methyl-1-oxacyclopentadec- | 40 |
| | | 10-en-2-one | |
| 1637294-12-2 | Nympheal | 3-(4-isobutyl-2-methylphenyl)propanal | 40 |
| 104-67-6 | Peach pure | 4-undecanolide | 40 |
| 916887-53-1 | Petalia | 2-cyclohexylidene-2-ortho-tolyl | 40 |
| | | acetonitrile | |
| 60-12-8 | Phenyl ethyl alcohol | 2-Phenylethanol | 40 |
| 104-61-0 | Prunolide | gamma-nonalactone | 60 |
| 28219-61-6 | Radjanol | 2-ethyl-4-(2,2,3-trimethyl-3- | 40 |
| | | cyclopenten-1-yl)-2-buten-1-ol | |
| 5471-51-2 | Raspberry ketone | 4-(4-hydroxyphenyl)-2-butanone | 20 |
| 477218-42-1 | Serenolide | cyclopropanecarboxylic acid 2-[1-(3,3- | 60 |
| | | dimethyl cyclohexyl)ethoxy]-2- | |
| | | methylpropyl ester | |
| 37609-25-9 | Velvione | 5-cyclohexadecen-1-one | 40 |
| | total | | 1000 |

This raspberry accord can be incorporated for example in home care applications, for room fragrancing, for example for use in candle at 4%. It gives a fruity gourmand overall impression, with a raspberry character, and a soft floral musky undertone. Its solubility in the mixture according to the invention is as good as in DPG.

| | 50% in DPG | 50% in 1:1 MIX |
|---|---|---|
| visual inspection | soluble | soluble |
| ultrasonic bath, spectrometer | 0.00 | 0.00 |

### b) Woody Accord

| | ingredient | | parts by |
|---|---|---|---|
| CAS-No. | name | chemical name | weight |
| 139504-68-0 | Amber | 1-(2-tert-butylcyclohexyl)oxybutan- | 300 |
| | core | 2-ol | |
| 4707-47-5 | Evernyl | Methyl 2,4-dihydroxy-3,6- | 100 |
| | | dimethylbenzoate | |
| 79-77-6 | Ionone | 4-(2,6,6-trimethyl-1-cyclohexen-1- | 200 |
| | beta | yl)-3-buten-2-one | |
| 70788-30-6 | Nimberol | 1-(2,2,6-trimethylcyclohexyl)hexan- | 100 |
| | | 3-ol | |
| 28219-61-6 | Radjanol | 2-ethyl-4-(2,2,3-trimethyl-3- | 300 |
| | | cyclopenten-1-yl)-2-buten-1-ol | |
| | total | | 1000 |

This woody accord can be incorporated for example in home care application, for room fragrancing, for example for use in candle at 4%. It gives a woody overall impression, with a cedarwood and sandalwood character. Its solubility in the mixture according to the invention is as good as in DPG.

| | 50% in DPG | 50% in 1:1 MIX |
|---|---|---|
| visual inspection | soluble | soluble |
| ultrasonic bath, spectrometer | 0.00 | 0.00 |

### Example 3: Solvent Mixtures with Water

### Perfume Accord:

| | | | parts by |
|---|---|---|---|
| CAS- No. | ingredient name | chemical name | weight |
| 93-29-8 | Acetyl isoeugenol crystals | (2-methoxy-4-prop-1-enylphenyl) | 20.00 |
| | | acetate | |
| 141-13-9 | Adoxal | 2,6,10-trimethyl-9-undecenal | 2.00 |
| 88-41-5 | Agrumex | 2-tert-butylcyclohexyl acetate | 800.00 |
| 124-13-0 | Aldehyde c 8 octylic food | Octanal | 1.00 |
| | grade | | |
| 2705-87-5 | Allyl cyclohexyl propionate | 2-propenyl 3-cyclohexylpropanoate | 60.00 |
| 142-19-8 | Allyl oenanthate | 2-propenyl heptanoate | 80.00 |
| 28645-51-4 | Ambrettolide | Oxacycloheptadec-10-en-2-one | 2.50 |
| 140-11-4 | Benzyl acetate | Benzyl acetate | 1000.00 |
| | Bitrex 10%/alc * | Lignocaine benzyl benzoate (10% in | 10.00 |
| | | EtOH) | |
| 106-22-9 | Citronellol extra | 3,7-dimethyl-6-octen-1-ol | 20.00 |
| 68901-15-5 | Cyclogalbanate | 2-propenyl (cyclohexyloxy)acetate | 5.00 |
| 23696-85-7 | Damascenone giv | 1-(2,6,6-trimethyl-1,3- | 0.50 |
| | | cyclohexadiene-1-yl)-2-butene-1- | |
| | | one | |
| 24720-09-0 | Damascone alpha | 1-(2,6,6-trimethyl-2-cyclohexen-1- | 1.00 |
| | | yl)-2-buten-1-one (e + z) | |
| 32480-08-3 | Dihydro farnesal | 3,7, 11-trimethyl-6, 10-dodecadienal | 22.00 |
| 18479-58-8 | Dihydro myrcenol | 2,6-dimethyl-7-octen-2-ol | 750.00 |
| 151-05-3 | Dimethyl benzyl carbinyl | 2-methyl-3-phenylpropan-2-yl | 100.00 |
| | acetate | acetate | |
| 67801-20-1 | Ebanol | 3-methyl-5-(2,2,3-trimethyl-3- | 20.00 |
| | | cyclopenten-1-yl)-4-penten-2-ol (& | |
| | | isomers) | |
| 7452-79-1 | Ethyl methyl-2-butyrate | Ethyl 2-methylbutanoate | 20.00 |
| 4707-47-5 | Evernyl | Methyl 2,4-dihydroxy-3,6- | 5.00 |
| | | dimethylbenzoate | |
| 125109-85-5 | Florhydral | 3-(3-isopropylphenyl)butanal | 25.00 |
| 63500-71-0 | Florosa | 4-methyl-2-(2- | 100.00 |
| | | methylpropyl)tetrahydro-2h-pyran-4- | |
| | | ol | |
| 6413-10-1 | Fructone | Ethyl 2-methyl-1,3-dioxolane-2- | 300.00 |
| | | acetate | |
| 24851-98-7 | Hedione | Methyl 3-oxo-2- | 100.00 |
| | | pentylcyclopentaneacetate | |
| | Hexenal-2-trans 10%/tec | Trans-2-hexenal (10% solution in | 5.00 |
| | | triethyl citrate) | |
| 3681-71-8 | Hexenyl-3-cis acetate | Cis-3-hexenyl acetate | 20.00 |
| 6259-76-3 | Hexyl salicylate | Hexyl 2-hydroxybenzoate | 200.00 |
| 79-77-6 | lonone beta | 4-(2,6,6-trimethyl-1-cyclohexen-1- | 100.00 |
| | | yl)-3-buten-2-one | |
| 32764-98-0 | Jasmolactone | 6-(pent-3-en-1-yl)tetrahydro-2h- | 1.50 |
| | | pyran-2-one | |
| 78-70-6 | Linalool synthetic | 3,7-dimethyl-1,6-octadien-3-ol | 2495.00 |
| 115-95-7 | Linalyl acetate synthetic | 3,7-dimethyl-1,6-octadien-3-yl | 500.00 |
| | | acetate | |
| 103694-68-4 | Majantol | 2,2-dimethyl-3-(3-methylphenyl)- | 50.00 |
| | | propanol | |
| 8008-57-9 | Orange oil colorless n.s. | Oils, orange, sweet | 50.00 |
| 104-67-6 | Peach pure | 4-undecanolide | 60.00 |
| 28219-61-6 | Radjanol super | 2-ethyl-4-(2,2,3-trimethyl-3- | 25.00 |
| | | cyclopenten-1-yl)-2-buten-1-ol | |
| | | (main component) | |
| 5471-51-2 | Raspberry ketone | 4-(4-hydroxyphenyl)-2-butanone | 40.00 |
| 16409-43-1 | Rose oxide co | 4-methyl-2-(2-methyl-1- | 10.00 |
| | | propenyl)tetrahydro-2h-pyran | |
| 65113-99-7 | Sandalore extra | 5-(2,2,3-trimethyl-3-cyclopentenyl)- | 25.00 |
| | | 3-methylpentan-2-ol | |
| 70788-30-6 | Timberol | Cyclohexanepropanol, 2,2,6- | 5.00 |
| | | trimethyl-alpha-propyl- | |
| 68039-49-6 | Tricyclal | 2,4-dimethyl-3-cyclohexene-1- | 45.00 |
| | | carbaldehyde (z)(main component) | |
| 81782-77-6 | Undecavertol | 4-methyl-3-decen-5-ol | 80.00 |
| 121-33-5 | Vanillin | 4-hydroxy-3-methoxybenzaldehyde | 2.00 |
| 37609-25-9 | Velvione | 5-cyclohexadecen-1-one | 2.50 |
| 101-48-4 | Viridine | Phenylacetaldehyde dimethyl acetal | 10.00 |
| | Ylang eco essence | Multi-component mixture | 30.00 |
| | total | | 7200 |

* Bitrex is a bitter and odorless compound that is added to a composition to prevent accidental swallowing.

The accord from the table above can be dissolved in the solvent mixture of the present invention and water.

| | | | |
|---|---|---|---|
| Sample | A | B | C |

composition (parts by weight)

| | | | |
|---|---|---|---|
| Water | 10 | 10 | 10 |
| perfume accord | 10 | 20 | 30 |
| MIX 1:1 | 80 | 70 | 60 |
| total | 100 | 100 | 100 |

solubility assesment

| | | | |
|---|---|---|---|
| visual inspection | soluble | soluble | soluble |
| Vortex, visual | soluble | soluble | non |
| inspection | | | soluble |
| ultrasonic bath, | 0.04 | 0.03 | >2 |
| spectrometer | | | |

In a perfume composition, in which water is present at 10%, the solvent mixture of the present invention (MIX 1:1=1,3-butylene glycol and triethyl citrate in a ratio 1:1) can dissolve the accord described above in good amounts. The overall composition may contain up to 20% or more, for example 25% by weight of the accord in a solution containing 10% water and an amount of the solvent mixture of the present invention (MIX 1:1) adding up to 100% (Samples A and B). A perfume composition with 30% by weight of the perfume accord (Sample C) cannot be fully solubilized. It can be concluded that the solvent mixture of the present invention allows the incorporation of the accord into a consumer product which base is containing water.

### Example 4: Miscibility and Stability in Matrix and Application

The miscibility and stability of the solvent mixture of the present invention was compared with those of DPG and the pure mixture ingredients. The miscibility with isopropyl myristate (IPM) and ethylene brassylate (EB) mimics the miscibility with a fragrance oil matrix. The miscibility with the Eau de Toilette (EDT) premix (75.86% ethanol and 14.14% water) at different concentrations mimics the miscibility with ethanol/water at the dosage level typical for EDT. Each sample was rated right after preparation (t=0 at room temperature), and after 14 days at −10° C., 5° C. and 60° C., and after 24 h exposure to UV light. The results are summarized in the table below.

| | | | | | 10% in | 5% in | 3% in |
|---|---|---|---|---|---|---|---|
| | | | 10% in | 10% in | EDT | EDT | EDT |
| | conditions | pure | IPM | EB | premix | premix | premix |
| DPG | t = 0 | clear, slightly | turbid first, | clear | clear | clear | clear |
| | | greasy smell | clear after | | | | |
| | | | stirring | | | | |
| | −10° C. | | | | clear | clear | clear |
| | 5° C. | | biphasic | clear | clear | clear | clear |
| | 14 d 60° C. | | biphasic | clear | clear | clear | clear |
| | 24 h UV | | biphasic | clear | clear | clear | clear |
| TEC | t = 0 | clear, slight | nearly clear, | nearly clear, | clear | clear | clear |
| | | greasy smell | slightly hazy | slightly hazy | | | |
| | −10° C. | | | | clear | clear | clear |
| | 5° C. | | clear | clear | clear | clear | clear |
| | 14 d 60° C. | | clear | clear | clear | clear, | clear, |
| | | | | | | medium | medium |
| | | | | | | solvent | solvent |
| | | | | | | scent | scent |
| | 24 h UV | | clear | clear | clear | clear | clear |
| 1,3- | t = 0 | colorless | not miscible: | not miscible: | clear | | |
| butylene | | liquid, | very turbid/ | very turbid/ | | | |
| glycol | | strong smell | opaque | opaque | | | |
| | | (burnt latex) | | | | | |
| | 5° C. | clear | biphasic, hazy | almost opaque, | clear | | |
| | | | | white, biphasic | | | |
| | 14 d 60° C. | clear, very | biphasic, | almost opaque, | clear, very | | |
| | | strong burnt | hazy, strong | white, biphasic | strong | | |
| | | candle smell | gas/burnt | | gas/burnt | | |
| | | | candle smell | | candle smell | | |
| | 24 h UV | clear, | biphasic, | almost opaque, | clear | | |
| | | odor conform | 1,3-butylene | white, biphasic | | | |
| | | to control | glycol at | | | | |
| | | | the bottom, | | | | |
| | | | strong | | | | |
| | | | gas/burnt | | | | |
| | | | candle smell | | | | |
| Mix | t = 0 | clear | Some | clear | clear | | |
| TEC/1,3- | | | difficulties in | | | | |
| butylene | | | incorporating | | | | |
| glycol | | | the mix, clear | | | | |
| 80/20 | | | after 5 min | | | | |
| | | | agitation | | | | |
| | | | clear | | | | |
| | −10° C. | | | | clear | | |
| | 5° C. | clear | small crystals | clear | clear | | |
| | | | at the bottom | | | | |
| | 14 d 60° C. | clear, slight | clear | clear | clear | | |
| | | acceptable | | | | | |
| | | difference | | | | | |
| | | in odor | | | | | |

By the comparison it is demonstrated, that the solvent mixture of the present invention shows similar miscibility and stability properties as DPG in a fragrance oil matrix as well as in consumer products like Eau de Toilette (at 10%), and could therefore serve as a replacer for DPG. In contrast, the individual materials 1,3-butylene glycol and triethyl citrate show disadvantages when compared to DPG.

### Example 5: Solubility of Fragrance Ingredients in TEC/1,3-Butylene Glycol-80/20—Mixture

| Entry | Sample | t = 0 | 14 d 5° C. | 14 d 60° C. |
|---|---|---|---|---|
| 1 | 10% vanillin in | 15-20 min stirring | OK | OK |
| | MIX 80/20 | at room temperature | | |
| 2 | 15% vanillin in | 5 min. heating at | OK | OK |
| | MIX 80/20 | 50° C. + 8-10 hrs. | | |
| | | stirring at room | | |
| | | temperature | | |
| | | Solubility OK | | |
| 3 | 20% vanillin in | 5 min. heating at | OK | OK |
| | MIX 80/20 | 50° C. + 8-10 hrs. | | |
| | | stirring at room | | |
| | | temperature | | |
| | | Solubility OK | | |
| 4 | 25% vanillin in | 5 min. heating at | OK | OK |
| | MIX 80/20 | 50° C. + 8-10 hrs. | | |
| | | stirring at room | | |
| | | temperature | | |
| | | Solubility OK | | |
| 5 | 0.5% Tonka | OK | OK | OK |
| | roasted 30% in | | | |
| | EtOH in MIX | | | |
| | 80/20 | | | |
| 6 | 1% Tonka | OK | OK | OK |
| | roasted 30% in | | | |
| | EtOH in MIX | | | |
| | 80/20 | | | |
| 7 | 1.5% Tonka | OK | OK | OK |
| | roasted 30% in | | | |
| | EtOH in MIX | | | |
| | 80/20 | | | |
| 8 | 2% Tonka | OK | OK | OK |
| | roasted 30% in | | | |
| | EtOH in MIX | | | |
| | 80/20 | | | |

MIX 80/20: Mix TEC/1,3-butylene glycol—80/20

In the table above, the solubility of single ingredients solubility has been summarized. Different ingredients have been assessed for their solubility in a mixture of TEC/1,3-butylene glycol at 80:20 at different concentrations. Furthermore, the storage stability at 5 and 60° C. for 14 days have been tested.

### Example 6: Vanillin Solubility in Other Solvents

| | 1 D | 1 D | 14 d | 14 d |
|---|---|---|---|---|
| | 5° C. | 60° C. | 5° C. | 60° C. |
| 10% in DPG | OK | OK | OK | OK, yellow |
| | | | | coloration |
| 15% in DPG | OK | OK | OK | OK, yellow |
| | | | | coloration |
| 20% in DPG | OK | OK | OK | OK, yellow |
| | | | | coloration |
| 10% in TEC | OK | OK | OK | OK, light yellow |
| | | | | coloration |
| 15% in TEC | some cristals | OK | recristalisation | OK, light yellow |
| | at the bottom | | non reversible | coloration |
| | | | @RT without | |
| | | | stirring | |

It can be seen from the table above and the results of the table of example 5, that vanillin, a crystalline polar compound, can be well dissolved in an amount of at least 25% in the solvent mixture of the present invention. The amount that can be dissolved in DPG is about 20%, when saturation occurs. The compound can be dissolved at less than 15% in triethylcitrate (TEC).

1. A solvent mixture consisting of:
a) 1,3-butylene glycol, and
b) triethyl citrate.

2. A solvent mixture comprising:
a) 1,3-butylene glycol, and
b) triethyl citrate.

3. The solvent mixture according to claim 1, wherein the ratio of 1,3-butylene glycol and triethyl citrate is between 1:5 and 5:1.

4. The solvent mixture according to claim 1, wherein the ratio of 1,3-butylene glycol and triethyl citrate is between 1:4 and 4:1.

5. The solvent mixture according to claim 1, wherein the ratio of 1,3-butylene glycol and triethyl citrate is between 1:3 and 3:1.

6. The solvent mixture according to claim 1, wherein the ratio of 1,3-butylene glycol and triethyl citrate is 1:1.

7. The solvent mixture according to claim 1 further comprising water.

8. (canceled)

9. (canceled)

10. A fragrance composition comprising one or more fragrance compounds and the solvent mixture according to claim 1.

11. The fragrance composition according to claim 10, wherein the composition is essentially free of dipropylene glycol (DPG).

12. The fragrance composition according to claim 11, consisting essentially of one or more fragrance compounds and the solvent mixture according to claim 1 and ethanol.

13. A method of utilizing the solvent mixture according to claim 1 as solvent for a fragrance compound and/or fragrance composition, wherein the method comprises adding the solvent mixture to the fragrance compound and/or fragrance composition.

14. A method of replacing dipropylene glycol (DPG) in a fragrance composition, wherein the method comprises replacing DPG with the solvent mixture according to claim 1.

15. A method for perfuming consumer products selected from the group consisting of fine fragrance, personal care products, fabric care products, home care products and air care products, wherein the method comprises adding the fragrance composition according to claim 10 to the consumer products.

16. A consumer product comprising the fragrance composition according to claim 10 and a consumer product base.

17. The consumer product according to claim 16, wherein the consumer product base contains water.

18. A method to confer, enhance, improve or modify the hedonic properties of a consumer product, the method comprising the addition of the fragrance composition according to claim 10 to said consumer product.

19. The solvent mixture according to claim 2, wherein the ratio of 1,3-butylene glycol and triethyl citrate is between 1:5 and 5:1.

20. The solvent mixture according to claim 2 further comprising water.

21. A fragrance composition comprising one or more fragrance compounds and the solvent mixture according to claim 2.

22. A method to confer, enhance, improve or modify the hedonic properties of a consumer product, the method comprising the addition of the fragrance composition according to claim 21 to said consumer product.

23. A method of utilizing the solvent mixture according to claim 2 as solvent for a fragrance compound and/or fragrance composition, wherein the method comprises adding the solvent mixture to the fragrance compound and/or fragrance composition.

24. A method of replacing dipropylene glycol (DPG) in a fragrance composition, wherein the method comprises replacing DPG with the solvent mixture according to claim 2.
